(19) United States
(12) Patent Application Publication
(10) Pub. No.: US 2024/0138833 A1
(43) Pub. Date: May 2, 2024

(54) RELOAD ASSEMBLY FOR SURGICAL STAPLING DEVICE

(71) Applicant: Covidien LP, Mansfield, MA (US)

(72) Inventors: Zhaokai Wang, Shanghai (CN); Xiliang Zhang, Shanghai (CN); Syed Sarfraz Ahamed, Shanghai (CN)

(21) Appl. No.: 18/280,619

(22) PCT Filed: Mar. 5, 2021
(86) PCT No.: PCT/CN2021/079220

§ 371 Date: Sep. 6, 2023

(51) Int. Cl. A61B 17/072

(52) U.S. Cl. CPC A61B 17/072; A61B 2017/07221; A61B 2017/07257; A61B 2017/07271; A61B 2017/07285

(57) ABSTRACT

A surgical stapling device includes an end effector frame and a replaceable reload assembly. The reload assembly includes a reload frame, a cartridge assembly, and an anvil assembly. The reload frame includes a longitudinal portion and a transverse portion that supports the anvil assembly. The longitudinal portion of the reload frame is engaged with the cartridge assembly via interlocking structure that facilitates movement of the cartridge assembly in relation to the anvil assembly between spaced and clamped positions. The reload frame is configured to be received on the end effector frame to properly position the cartridge and anvil assemblies in relation to each other.

### FIELD

This technology is generally related to surgical stapling devices and, more particularly, to surgical stapling devices with replaceable reload assemblies.

### BACKGROUND

Surgical stapling devices are commonly used during a variety of surgical procedures to expedite dissection and suturing of tissue and minimize trauma to a patient. Typically, these stapling devices include an end effector having a cartridge assembly and an anvil assembly that are movable in relation to each other between open and clamped positions. The cartridge assembly is configured to be removable from the end effector and replaceable after each use of the stapling device to facilitate reuse of the stapling device. The cartridge assembly also includes a knife assembly for cutting tissue clamped between the anvil and cartridge assemblies.

The anvil assembly supports a cutting plate, and the knife assembly includes a knife blade that has a cutting edge which is driven through tissue clamped between the anvil and cartridge assemblies and into the cutting plate of the anvil assembly to transect or resect the tissue. Typically, the cartridge assembly includes structure that confines the tissue between the cartridge and anvil assemblies and positions the tissue for engagement by the cutting edge of the knife blade.

A continuing need exists in the art for a surgical stapling device that includes an end effector including replaceable anvil and cartridge assemblies to facilitate improved transection and resection of tissue.

### SUMMARY

This disclosure is directed to a surgical stapling device that includes an end effector frame and a replaceable reload assembly. The reload assembly includes a reload frame, a cartridge assembly, and an anvil assembly. The reload frame includes a longitudinal portion and a transverse portion. The transverse portion of the reload frame supports the anvil assembly. The longitudinal portion of the reload frame is engaged with the cartridge assembly via interlocking structure that facilitates movement of the cartridge assembly in relation to the anvil assembly between spaced and clamped positions. The reload frame is configured to be received on the end effector frame to properly position the cartridge and anvil assemblies in relation to each other.

Aspects of this disclosure are directed to a reload assembly that includes a reload frame, an anvil assembly, and a cartridge assembly. The reload frame includes a longitudinal portion and a transverse portion. The longitudinal portion has a proximal end portion and a distal end portion. The transverse portion extends from the distal end portion of the longitudinal portion and has an outer surface and an inner surface. The anvil assembly is secured to the inner surface of the transverse portion and includes an anvil that defines a knife slot and staple forming depressions positioned on opposite sides of the knife slot. The cartridge assembly includes a cartridge body, a pusher, a knife assembly, and staples. The cartridge body defines a cavity, a knife slot, and staple receiving pockets positioned on each side of the knife slot. The pusher and the knife assembly are received within the cavity of the cartridge body and movable between retracted and advanced positions. The pusher includes fingers that are received within the staple receiving slots. One of the staples is received within each of the staple receiving slots such that movement of the pusher from its retracted position to its advanced position ejects the staples from the staple receiving slots. The cartridge body and the longitudinal portion of the reload frame include interlocking structure that couples the cartridge body to the longitudinal portion of the reload frame while facilitating longitudinal movement of the cartridge assembly in relation to the anvil assembly.

In aspects of the disclosure, the interlocking structure includes dove tail shaped protrusions on the cartridge body and correspondingly shaped channels defined in the longitudinal portion of the reload frame.

In some aspects of the disclosure, the interlocking structure includes rectangular protrusions on the cartridge body and correspondingly shaped channels defined in the longitudinal portion of the reload frame.

In certain aspects of the disclosure, the longitudinal portion of the reload frame includes an inner surface and an outer surface, and the inner surface has a raised rib that extends inwardly from the inner surface of the longitudinal portion and is received within a channel defined by the cartridge body.

In aspects of the disclosure, the raised rib and the channel defined by cartridge body have stepped configurations.

In some aspects of the disclosure, the anvil assembly includes a cut plate that is sandwiched between the transverse portion of the reload frame and the anvil.

In certain aspects of the disclosure, the anvil includes resilient brackets and the transverse portion of the reload frame includes slots, and the resilient brackets are received within the slots to secure the anvil and the cut plate onto the inner surface of the transverse portion of the reload frame.

In aspects of the disclosure, the transverse portion of the reload frame includes a tab that is positioned within each of the slots, and each of the resilient brackets defines an opening that receives one of the tabs to secure the anvil and the cut plate to the reload frame.

In some aspects of the disclosure, the cut plate includes a rib that is aligned with the knife slot of the anvil.

In certain aspects of the disclosure, the rib extends through the knife slot of the anvil.

In aspects of the disclosure, the cut plate includes a proximal extension that extends proximally from the cut plate to a position proximally of the anvil and the knife assembly includes a cutting blade.

In some aspects of the disclosure, the extension is positioned on one end of the anvil and includes an inner surface that is angled inwardly to a position inwardly of an end of the knife slot.

In certain aspects of the disclosure, the extension is positioned to engage the cutting blade of the knife assembly when the knife assembly is moved to its advanced position.

In aspects of the disclosure, the cut plate is formed from polypropylene and the anvil is formed from stainless steel.

In some aspects of the disclosure, the outer surfaces of the longitudinal and transverse portions of the reload frame define recesses that are configured receive a portion of a surgical device.

Other aspects of the disclosure are directed to a surgical stapling device including an elongate frame, a thrust bar, a clamp slide assembly, and an end effector. The elongate frame has a proximal portion and a distal portion and defines a channel. The thrust bar has a distal portion, is positioned within the channel, and is movable between a retracted position and an advanced position. The clamp slide assembly is positioned within the channel and has a proximal portion and a distal portion. The distal portion defines a clamp slide channel. The clamp slide assembly is movable within the channel defined by the elongate frame between retracted and advanced positions. The distal portion of the thrust bar is positioned within the clamp slide channel. The end effector frame is secured to the distal portion of the elongate frame and includes a first transverse portion, a second transverse portion, and a longitudinal portion. The longitudinal portion interconnects the first transverse portion and the second transverse portion at spaced positions to define a gap between the first and second transverse portions. The distal portion of the clamp slide assembly is positioned within the gap. The reload assembly includes a reload frame, an anvil assembly, and a cartridge assembly. The reload frame is releasably supported on the end effector frame and includes a longitudinal portion and a transverse portion. The longitudinal portion of the reload frame has a proximal end portion and a distal end portion. The transverse portion of the reload frame extends from the distal end portion of the longitudinal portion of the reload frame and has an outer surface and an inner surface. The anvil assembly is secured to the inner surface of the transverse portion of the reload frame and includes an anvil that defines a knife slot and staple forming depressions positioned on opposite sides of the knife slot. The cartridge assembly includes a cartridge body, a pusher, a knife assembly, and staples. The cartridge body defines a cavity, a knife slot, and staple receiving pockets positioned on each side of the knife slot. The pusher and the knife assembly are received within the cavity of the cartridge body and movable between retracted and advanced positions. The pusher includes fingers that are received within the staple receiving slots. One of the staples is received within each of the staple receiving slots such that movement of the pusher from its retracted position to its advanced position ejects the staples from the staple receiving slots. The cartridge body and the longitudinal portion of the reload frame include interlocking structure that couples the cartridge body to the longitudinal portion of the reload frame while facilitating longitudinal movement of the cartridge assembly in relation to the anvil assembly between a retracted position and an advanced position.

In aspects of the disclosure, the transverse portion of the reload frame includes an outer surface that defines a recess that receives the first transverse portion of the end effector frame when the reload assembly is loaded onto the end effector frame.

In some aspects of the disclosure, the first transverse portion of the end effector frame includes a central portion and defines longitudinal channels on each side of the central portion.

In certain aspects of the disclosure, the central portion of the first transverse portion of the end effector frame is received within the recess of the transverse portion of the reload frame.

In aspects of the disclosure, the knife assembly includes a knife holder that define a slot and a cutting blade.

In some aspects of the disclosure, the distal portion of the thrust bar includes spaced protrusions that are received within the slot of the knife holder when the reload assembly is loaded onto the end effector frame such that movement of the thrust bar from its advanced position towards its retracted position moves the knife assembly from its advanced position towards its retracted position.

In certain aspects of the disclosure, the distal portion of the thrust bar includes spaced force distribution members and the knife holder includes an abutment surface, and the spaced force distribution members are positioned along the distal portion of the thrust bar in engagement with the abutment surface of the thrust bar to balance firing forces applied by the thrust bar on the knife holder.

In aspects of the disclosure, the cartridge body is secured to a housing, and the housing is at least partly received within the clamp slide channel when the reload assembly is loaded onto the end effector frame.

In some aspects of the disclosure, the distal portion of the clamp slide assembly includes ribs positioned within the clamp slide channel, and the housing defines transversely extending slots.

In aspects of the disclosure, the transversely extending slots receive the ribs when the reload assembly is loaded onto the end effector frame.

Other features of the disclosure will be appreciated from the following description.

### BRIEF DESCRIPTION OF THE DRAWINGS

Various aspects of the disclosure are described herein below with reference to the drawings, wherein:

FIG. 1 is a side perspective view of a surgical stapling device according to aspects of the disclosure including an end effector in an open position;

FIG. 2 is an enlarged view of the indicated area of detail shown in FIG. 1;

FIG. 3 is a side perspective view of the end effector shown in FIG. 1 with a reload assembly of the end effector separated from a frame of the end effector;

FIG. 3A is a side perspective view of distal portions of a thrust bar and clamp slide assembly of the surgical stapling device shown in FIG. 1;

FIG. 4 is a side perspective view of the reload assembly of the end effector shown in FIG. 3;

FIG. 5 is a perspective view from a proximal end of the reload assembly shown in FIG. 4;

FIG. 6 is an exploded, side perspective view of the reload assembly shown in FIG. 5;

FIG. 7 is an enlarged view of the indicated area of detail shown in FIG. 6;

FIG. 8 is side perspective view from the proximal end of a reload frame of the reload assembly shown in FIG. 6;

FIG. 9 is a cross-sectional view taken along section line 9-9 of FIG. 2;

FIG. 10 is cross-sectional view taken along section line 10-10 of FIG. 2 illustrating an alternate version of the end effector;

FIG. 11 is an enlarged view of the indicated area of detail shown in FIG. 6 illustrating an exploded view of an anvil assembly;

FIG. 12 is a side perspective view of a cutting plate of the anvil assembly shown in FIG. 11;

FIG. 13 is a cross-sectional view taken along section line 13-13 of FIG. 2;

FIG. 14 is a side perspective view from the proximal end of the reload assembly shown in FIG. 3;

FIG. 15 is a cross-sectional view taken along section line 15-15 of FIG. 2;

FIG. 16 is a side perspective view of a knife assembly of the reload assembly shown in FIG. 6;

FIG. 17 is a side perspective view of a distal portion of a thrust bar of the surgical stapling device shown in FIG. 1;

FIG. 18 is a side perspective view of the knife assembly of the reload assembly shown in FIG. 3 as the knife assembly is engaged with the thrust bar of the stapling device shown in FIG. 1;

FIG. 19 is a side perspective view of the knife assembly and thrust bar shown in FIG. 18 with the knife assembly coupled to the thrust bar;

FIG. 20 is a cross-sectional view taken along section line 20-20 of FIG. 3;

FIG. 20A is a perspective view from the distal end of the clamp slide assembly shown in FIG. 3A;

FIG. 21 is a side perspective view of the reload assembly and the frame of the end effector as the reload assembly is being positioned on the frame;

FIG. 22 is a cross-sectional view taken along section line 22-22 of FIG. 2;

FIG. 23 is a cross-sectional view taken along section line 23-23 of FIG. 2;

FIG. 24 is a cross-sectional view taken along section line 24-24 of FIG. 2;

FIG. 25 is a cross-sectional view taken through a proximal portion of the end effector with the reload assembly supported on the frame of the end effector and the end effector in an open position;

FIG. 26 is a cross-sectional view taken along section line 26-26 of FIG. 2;

FIG. 27 is a side cross-sectional view of the end effector shown in FIG. 2 in the clamped position;

FIG. 28 is a cross-sectional view taken along section line 28-28 of FIG. 27;

FIG. 29 is a cross-sectional cutaway view of a portion of the end effector shown in FIG. 27 with the end effector in the clamped and fired position; and

FIG. 30 is a side perspective view of the end effector shown in FIG. 27 coupled to the thrust bar after the end effector has been fired, as the end effector is moved back towards the open position, with all the components of the end effector shown in phantom except for the knife assembly.

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the stapling device in its customary manner, while the term “distal” is used generally to refer to that portion of the device that is farther from the clinician during use of the stapling device in its customary manner. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

This disclosure is directed to a surgical stapling device that includes an end effector frame and a replaceable reload assembly. The reload assembly includes a reload frame, a cartridge assembly, and an anvil assembly. The reload frame includes a longitudinal portion and a transverse portion. The transverse portion of the reload frame supports the anvil assembly. The longitudinal portion of the reload frame is engaged with the cartridge assembly via interlocking structure that facilitates movement of the cartridge assembly in relation to the anvil assembly between spaced and clamped positions. The reload frame is configured to be received on the end effector frame to properly position the cartridge and anvil assemblies in relation to each other and to securely couple the reload assembly to the end effector frame.

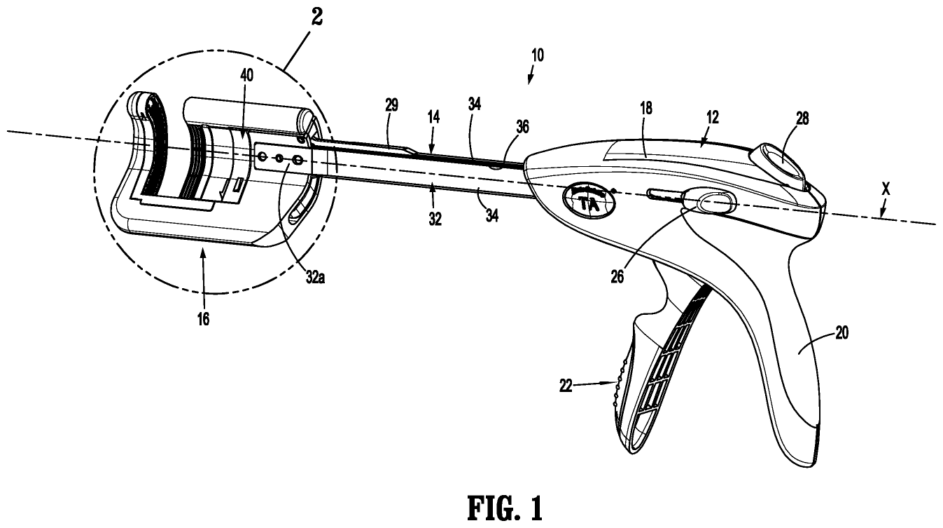

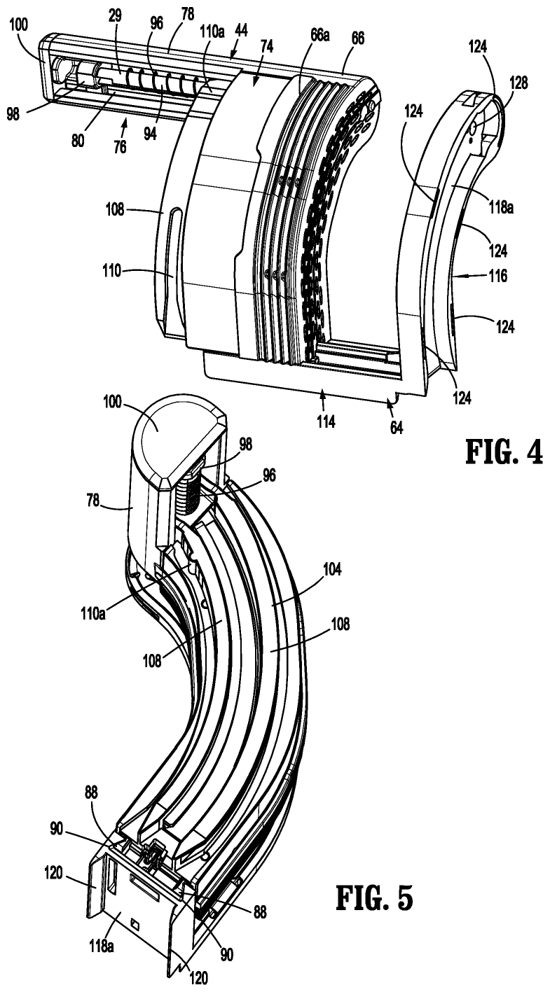

FIG. 1 illustrates a surgical stapling device according to aspects of the disclosure shown generally as stapling device 10. The stapling device 10 includes a handle assembly 12, an elongate body 14 that extends distally from the handle assembly 12, and an end effector 16 that is supported on a distal portion of the elongate body 14. The elongate body 14 defines a longitudinal axis “X”. The handle assembly 12 includes a housing 18 that defines a stationary handle 20 and supports a movable trigger 22. In aspects of the disclosure, the trigger 22 is supported by the housing 18 to pivot towards the stationary handle 20 between non-actuated and actuated positions to actuate the end effector 16, i.e., move the end effector 16 between open and clamped positions and fire the stapling device 10. The handle assembly 12 also supports two buttons 26 (only one is shown), one being positioned on each side of the housing 18, that are movable along the housing 18 to advance and retract an alignment pin pusher 29 (FIG. 4). The handle assembly 12 also includes a release button 28 that can be depressed to move the end effector 16 from the clamped position to the open position. For a more detailed description of a representative handle assembly 12, see, e.g., U.S. Pat. No. 6,817,508 (“the '508 Patent”).

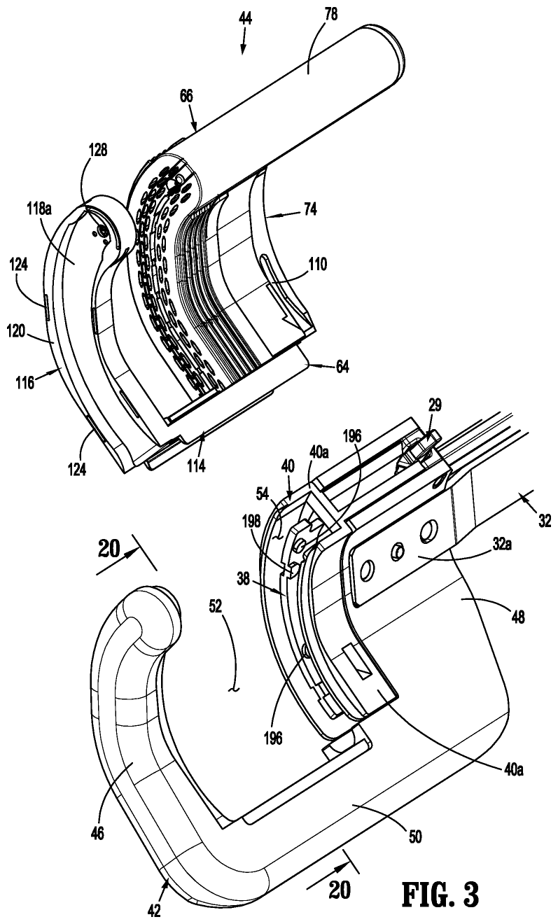

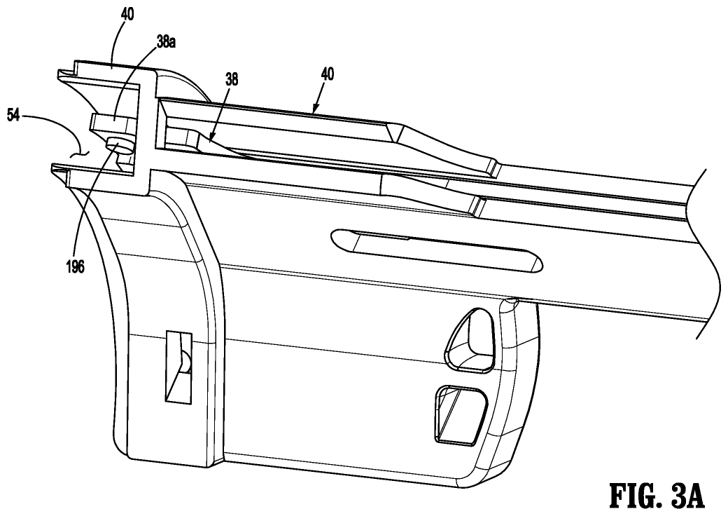

The stapling device 10 includes a frame 32 that extends from the handle assembly 12 and includes a distal portion 32a that supports the end effector 16. The frame 32 includes spaced frame members 34 that define a channel 36 that receives a thrust bar 38 (FIG. 17) and the alignment pin pusher 29. The thrust bar 38 (FIG. 17) is movable within the channel 36 between retracted and advanced positions in response to movement of the trigger 22 to actuate the stapling device 10. The channel 36 defined by the frame members 34 also receive a clamp slide assembly 40 (FIG. 3A) that extends into the end effector 16 as described in further detail below. For a more detailed description of the interaction between the trigger 22, the thrust bar 38, and the clamp slide assembly 40, see the '508 Patent.

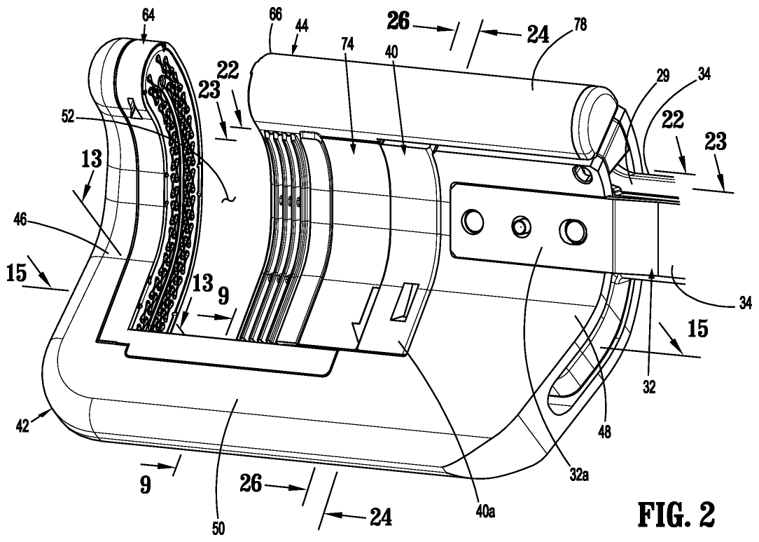

FIGS. 2-5 illustrate the end effector 16 which includes an end effector frame 42 and a reload assembly 44. The end effector frame 42 is secured to the distal portion 32a of the frame 32 of the stapling device 10 (FIG. 1) and includes a first transverse portion 46, a second transverse portion 48, and a longitudinal portion 50 that interconnects the first transverse portion 46 and the second transverse portion 48. The first and second transverse portions 46, 48 are spaced from each other to define a gap 52 between the first and second transverse portions 46, 48. In some aspects of the disclosure, the first and second transverse portions 46, 48 are curved along axes transverse to the longitudinal axis “X” of the elongate body 14 of the stapling device 10. In some aspects of the disclosure, the first and second transverse portions 46, 48 are formed from a plurality of linear portions, e.g., three, that are positioned at angles in relation to each other.

The clamp slide assembly 40 includes a proximal portion (not shown) that is coupled to the handle assembly 12 and a distal portion 40a (FIG. 3A) that is received within the gap 52 defined between the first and second transverse portions 46, 48 of the end effector frame 42. The distal portion 40a of the clamp slide assembly 40 defines a cavity 54. The distal portion 38a of the thrust bar 38 is received within the cavity 54 when the clamp slide assembly 40 and the thrust bar are in retracted positions. The cavity 54 receives the reload assembly 44 to releasably secure the reload assembly 44 to the end effector frame 42 and couple the thrust bar 38 to the reload assembly 44 as described in further detail below.

FIGS. 3-7 illustrate the reload assembly 44 which includes an anvil assembly 60, a cartridge assembly 62, and a reload frame 64. The anvil assembly 60 and the cartridge assembly 62 are coupled to the reload frame 64 to facilitate movement of the cartridge assembly 62 in relation to the anvil assembly 60 between the open and clamped positions as described in further detail below.

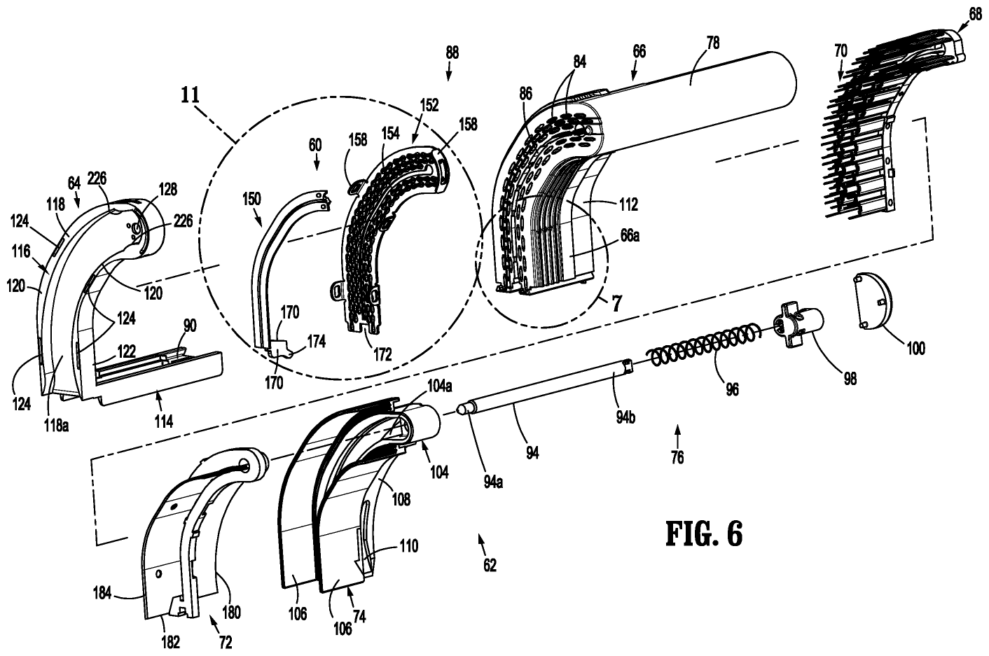

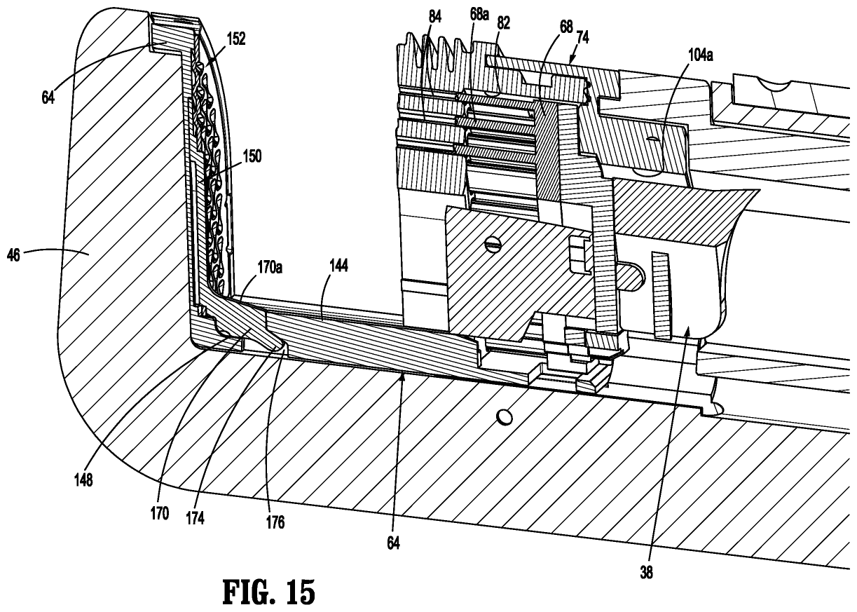

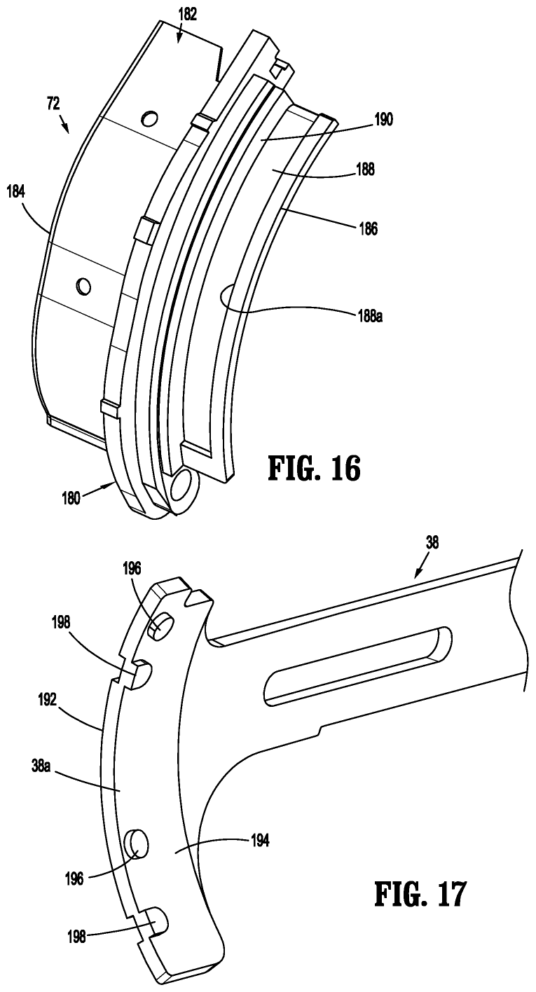

The cartridge assembly 62 includes a cartridge body 66, a pusher 68 (FIG. 6), staples 70, a knife assembly 72, a housing 74, and an alignment pin assembly 76 (FIG. 6). The cartridge body 66 has a shape that corresponds to the shape of the first transverse portion 48 of the end effector frame 42 and is formed from three linear sections that are angled in relation to each other. The cartridge body 66 includes a body portion 66a, and a proximal extension 78 that is positioned on one side of the body portion 66a and defines a longitudinally extending bore 80 (FIG. 4) that receives the alignment pin assembly 76. The body portion 66a of the cartridge body 66 defines a cavity 82 (FIG. 15) that has an open proximal end, a plurality of staple receiving slots 84, and a knife slot 86. In aspects of the disclosure, the staple receiving slots 84 are aligned in rows on opposite sides of the knife slot 86. Alternately, the staple receiving slots 84 may be formed in the body portion 66a of the cartridge body 66 in a variety of different configurations. Each of the staples 70 is received in a respective one of the staple receiving slots 84 of the body portion 66a. The cavity 82 of the body portion 66a receives the pusher 68 and the knife assembly 72 and is dimensioned to allow movement of the pusher 68 and the knife assembly 72 within the body portion 66a between retracted and advanced positions. The body portion 66a has a side spaced from the proximal extension 78 that is configured to engage the reload frame 64 in interlocking fashion to secure the cartridge body 66 to the reload frame 64. In aspects of the disclosure, the side of the body portion 66a of the cartridge body 66 spaced from the proximal extension 78 interlocking structure, e.g., dove tail protrusions 88 (FIG. 16), that are received in correspondingly shaped channels 90 in the reload frame 64 to secure the cartridge assembly 62 to the reload frame 64 while allowing longitudinal movement of the cartridge assembly 62 along the reload frame 64.

The pusher 68 is positioned within the cartridge body 66 distally of the knife assembly 72 and includes fingers 68a (FIG. 15) that are received within the staple receiving slots 84 of the cartridge body 66. The knife assembly 72 is positioned in abutting relation to the pusher 68 such that movement of the knife assembly 72 within the cavity 82 of the cartridge body 66 causes movement of the pusher 68 within the cavity 82 to eject the staples 70 from the staple receiving slots 84.

The alignment pin assembly 76 is received within in the longitudinal bore 80 in the proximal extension 78 of the cartridge body 66 and includes an alignment pin 94, a biasing member 96, a coupling member 98, and a cap 100. The alignment pin 94 has a distal end 94a and a proximal end 94b. The coupling member 98 is fixedly secured to the proximal end 94b of the alignment pin 94 and is biased proximally by the biasing member 96 to urge the alignment pin 94 proximally within the longitudinal bore 80 of cartridge body 66. As is known in art, the alignment pin 94 is movable distally by the alignment pin pusher 29 (FIG. 1) through openings in the pusher 90, knife assembly 72, housing 74, and cartridge body 66, into engagement with the anvil assembly 60 to confine tissue within the gap 42 of the end effector frame 42 and to guide movement of the cartridge assembly 62 towards the anvil assembly 60.

The housing 74 of the cartridge assembly 62 has a curvature that corresponds to the curvature of the cartridge body 66 and includes a curved base member 104 and curved side walls 106 that extend distally from the curved base member 104. In some aspects of the disclosure, the housing 74 is integrally formed with the cartridge body 66 and may form a portion of the cartridge body 66. The base member 104 includes side walls 108 that define a curved channel 104a (FIG. 15) that extends through the base member 104 from the distal end of the base member 104 to the proximal end of the base member 104. The channel 104a has an open distal end that communicates with the open proximal end of the cavity 82 (FIG. 15) of the cartridge body 66 and an open proximal end that facilitates passage of the clamp slide assembly 40 and the thrust bar 38 into the cartridge body 66. One or both of the side walls 108 of the base member 104 includes an outer surface that defines a transversely extending slot 110 (FIG. 6) and includes a protrusion 110a (FIG. 4). The side walls 106 of the housing 74 are received over and secured to a proximal portion 112 of the body portion 66a of the of the cartridge body 66 such that the cavity 82 of the cartridge body 66 is aligned with the channel 104a of the housing 104.

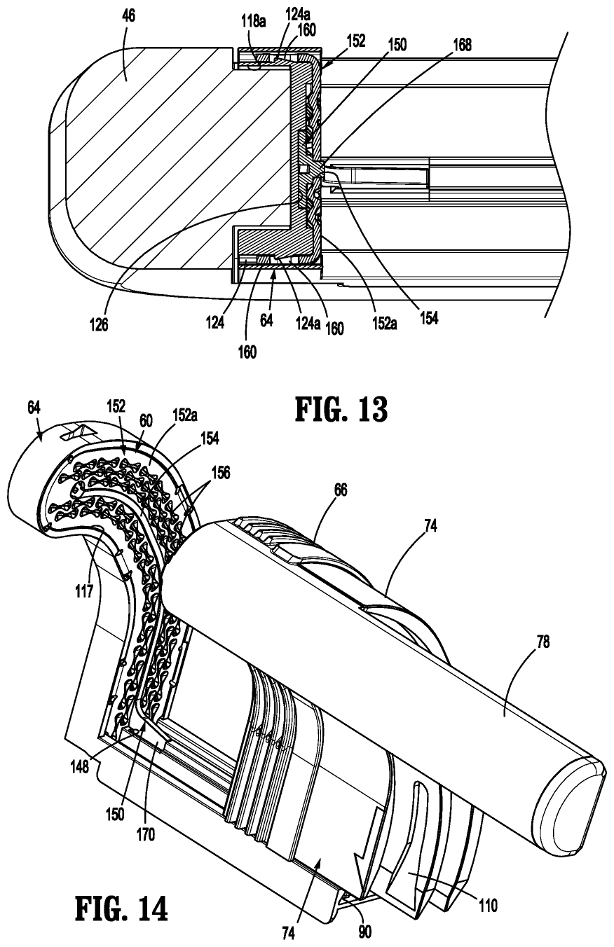

FIGS. 6 and 8 illustrate the reload frame 64 which includes a longitudinal portion 114 and a transverse portion 116 that is substantially perpendicular to the longitudinal portion 114. The transverse portion 116 includes an inner surface 117 (FIG. 17) and an outer surface 118 (FIG. 6). The outer surface 118 includes side walls 120 that define a curved recess 118a that receives a portion of the first transverse portion 46 (FIG. 3) of the end effector frame 42 when the reload 44 is loaded onto the end effector 42. The side walls 120 define slots 124 that extend through the reload frame 64 from the outer surface 118 to the inner surface 117. Tabs 124a (FIG. 13) are formed within the slots 124 and engage the anvil assembly 60 as described below to secure the anvil assembly 60 to the reload frame 64. The inner surface 114 of the transverse portion 114 of the reload frame 64 includes side walls 122 that define a curved, stepped recess 126 that receives the anvil assembly 60. The transverse portion 116 of the reload frame 64 defines an opening 128 that receives the alignment pin 94 of the alignment pin assembly 76 when the alignment pin 94 is moved from the retracted position to the advanced position.

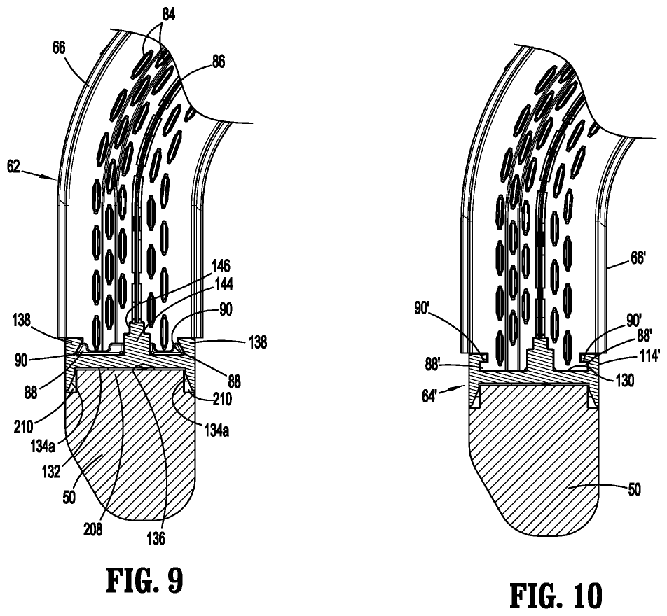

The longitudinal portion 114 of the reload frame 64 also includes an inner surface 130 (FIG. 8) and an outer surface 132. The outer surface 132 includes side walls 134 that defines a longitudinal recess 136 that receives the longitudinal portion 50 of the end effector frame 42 when the reload assembly 44 is loaded onto the end effector frame 42. In aspects of the disclosure, the side walls 134 include inner surfaces 134a that diverge outwardly (FIG. 9) in the direction of the longitudinal portion 50 of the end effector frame 42 to allow the reload frame 64 to self-align with the longitudinal portion 50 of the end effector frame 42 as the reload assembly 44 is loaded onto the end effector frame 42. The inner surface 130 of the longitudinal portion 114 of the reload frame 64 also includes side walls 138 that define a longitudinal recess 140 that receives one end of body portion 66a of the cartridge body 66. The side walls 138 define the dove tail shaped channels 90 which receive the dove tails 88 (FIG. 16) of the cartridge body 66 to secure the cartridge body 66 to the reload frame 64 for longitudinal movement between retracted and advanced positions.

The inner surface 130 of the longitudinal portion 114 of the reload frame 64 supports or includes a raised rib 144 that extends along a portion of the length of the longitudinal portion 114 and is received within a channel 146 (FIG. 9) defined in the cartridge body 66 to support the cartridge body 66 on the reload frame 64. In aspects of the disclosure, a cutout 148 (FIG. 8) is formed through the longitudinal portion 114 of the reload frame 64 at a position adjacent the transverse portion 116 of the reload frame 64 and in alignment with the raised rib 144. In other aspects of the disclosure, the raised rib 144 and the channel 146 are stepped to provide added support to the cartridge body 66 on the reload frame 64.

FIG. 10 illustrates an alternate version of the reload frame 64 and the cartridge body 66 shown generally as reload frame 64′ and cartridge body 66′. The reload frame 64, is substantially the same as the reload frame 64 except that the side walls 138′ of the inner surface 130′ of the longitudinal portion 114′ of the reload frame 64′ define rectangular shaped channels 90′. Similarly, the cartridge body 66′ is substantially the same as the cartridge body 66 except that the side of the cartridge body 66′ spaced from the proximal extension 78 (FIG. 2) includes rectangular protrusions 88′. As described above regarding the reload frame 64 and the cartridge body 66, the rectangular shaped channels 90′ receive the rectangular protrusions 88′ of the cartridge body 66′ to secure the cartridge body 66′ to the reload frame 64′ for longitudinal movement between retracted and advanced positions.

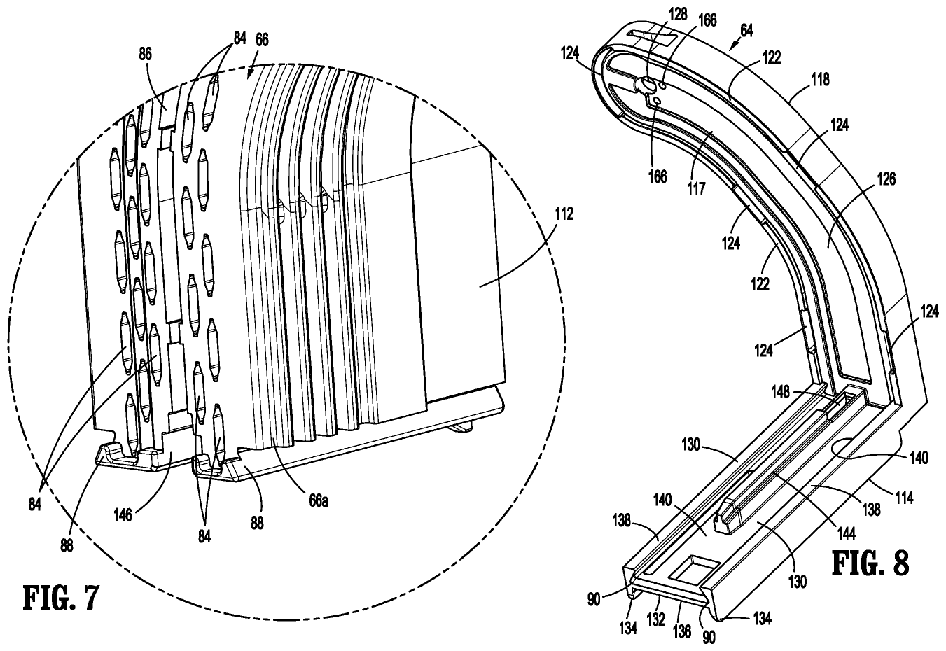

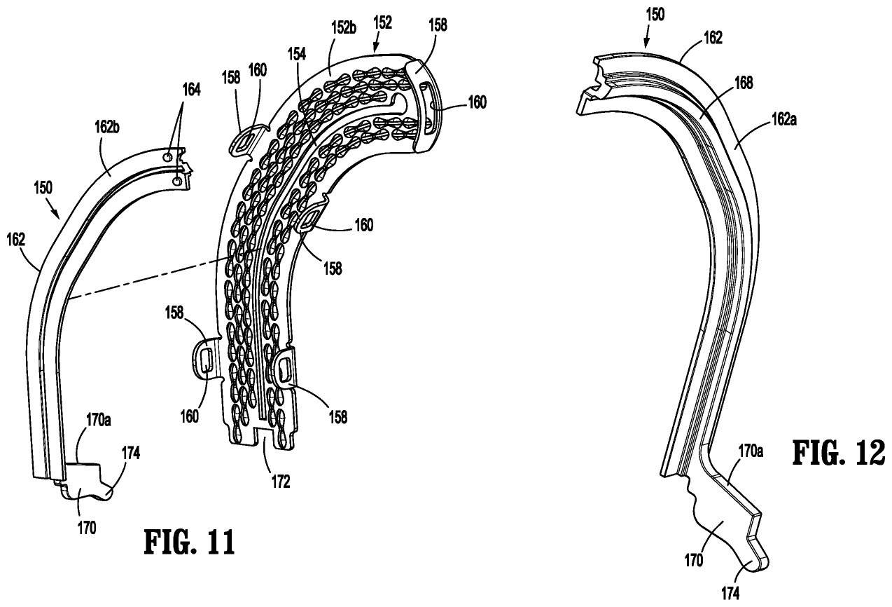

FIGS. 11-15 illustrate the anvil assembly 60 which includes a cut plate 150 and an anvil 152. The anvil 152 includes a distal surface 152b, a proximal surface 152a that defines a knife slot 154, and a plurality of staple forming depressions 156 (FIG. 7) positioned on each side of the knife slot 154. In aspects of the disclosure, the staple forming depressions 156 are aligned in rows on each side of the knife slot 154 and are positioned to align with the staple receiving slots 84 (FIG. 9) in the cartridge body 66 when the end effector 16 is in the clamped position. The anvil 152 includes resilient brackets 158 that extend proximally from the proximal surface 152a of the anvil 152 and define openings 160. The anvil 152 has a curved configuration and is received within the curved recess 126 (FIG. 8) defined in the inner surface 114 of the transverse portion 114 of the reload frame 64.

The cut plate 150 includes a curved body 162 that has a proximal surface 162a (FIG. 12), and a distal surface 162b (FIG. 11) that includes spaced protrusions 164. The cut plate 162 is received within the curved recess 126 defined in the inner surface 114 of the transverse portion 114 of the reload frame 64 and is sandwiched between the anvil 152 and the transverse portion 114 of the reload frame 64. When the cut plate 150 is received within the curved recess 126 (FIG. 8) of the reload frame 64, the spaced protrusions 164 of the cut plate 150 (FIG. 8) are received within openings 166 (FIG. 8) formed in the reload frame 64 to properly position the cut plate 162 in relation to the reload frame 64.

The distal surface 162a of the cut plate 162 includes a curved rib 168 that extends proximally from the distal surface 162a along the length of the cut plate 162 and is received within the knife slot 154 of the anvil 152 when the anvil 152 and the cut plate 162 are assembled to the reload frame 64. In aspects of the disclosure, the curved rib 168 extends through and proximally of the proximal surface 152a of the anvil 152. When the anvil 152 is positioned on the transverse portion 116 of the reload frame 64 over the cut plate 162, the resilient brackets 158 are received within the slots 124 of the reload frame 64. As the resilient brackets 158 pass through the slots 124, the resilient brackets 158 engage the tabs 124a of the reload frame 64 and flex outwardly as the pass over the tabs 124a. When the openings 160 in the resilient brackets 158 of the anvil 152 align with the tabs 124a, the resilient brackets 158 snap back to secure the anvil 152 and the cut plate 162 onto the reload frame 64.

The cut plate 162 includes an extension 170 that is formed on one end of the cut plate 162 and extends proximally through a notch 172 (FIG. 11) in the anvil 152. The extension 170 is aligned with the rib 168 and is received in the cutout 148 (FIG. 15) of the reload frame 150. The extension 170 includes a finger 174 that is received within a cavity 176 (FIG. 15) defined beneath a distal portion of the raised rib 144 (FIG. 15). Receipt of the extension 170 of the cut plate 150 within the cutout 148 of the reload frame 64 helps to secure and align the cut plate 162 with the reload frame 150. The extension 170 has an inner surface 170a that is aligned with the raised rib 144 and extends upwardly as viewed in FIG. 15 towards the anvil 152 to a position inwardly of an end of the knife slot 154. The inner surface 170a of the extension 170 is positioned to engage a cutting blade 182 of the knife assembly 72 adjacent an inner edge of the cutting blade 182 to ensure that tissue is cleanly cut through during a firing stroke of the stapling device 10.

FIGS. 16-19 illustrate the knife assembly 72 and the thrust bar 38. The knife assembly 72 includes a knife holder 180 and the cutting blade 182. The cutting blade 182 includes a proximal portion that is secured to the knife holder 180 and a distal portion that includes a cutting edge 184. The knife holder 180 includes a proximal portion 186 that defines a curved slot 188 and a curved abutment surface 190 that is positioned distally of the curved slot 188. The proximal end of the curved slot 188 is defined by a curved wall 188a.

The distal portion 38a of the thrust bar 38 is curved and includes a convex side 192 and a concave side 194. The concave side 194 supports spaced protrusions 196 and spaced force distribution members 198. The spaced protrusions 196 are received within the curved slot 188 when the reload assembly 44 is loaded onto the clamp slide assembly 40 (FIG. 3) and engage the curved wall 188a. Engagement of the spaced protrusions 196 and the curved wall 188a couples the thrust bar 38 to the knife holder 180 such that proximal movement of the thrust bar 38 causes proximal movement of the knife assembly 72.

The force distribution members 198 extend outwardly from the concave side 194 of the distal portion 38a of the thrust bar 38 and engage the curved abutment surface 190 of the knife holder 180 when the reload assembly 44 is loaded onto the clamp slide assembly 40. The force distribution members 198 redistribute the forces applied to the knife driver 72 when the stapling device 10 (FIG. 1) is fired to compensate for the different shapes of the thrust bar 38 and the cartridge body 66 (FIG. 3). More specifically, as described above, the cartridge body 66 is formed of linear sections that are angled with respect to each other to form a curved cartridge body 66. In contrast, the distal portion 38a of the thrust bar 38 includes a single curved member. This variation in shape of the distal portion 38a of the thrust bar 38 and the cartridge body 66, absent the spaced force distribution members 198, may result in uneven firing forces along the pusher 68.

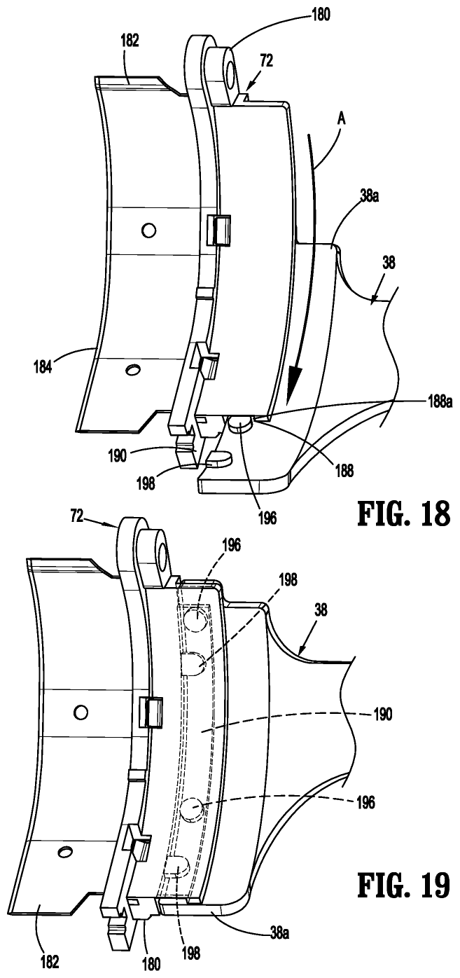

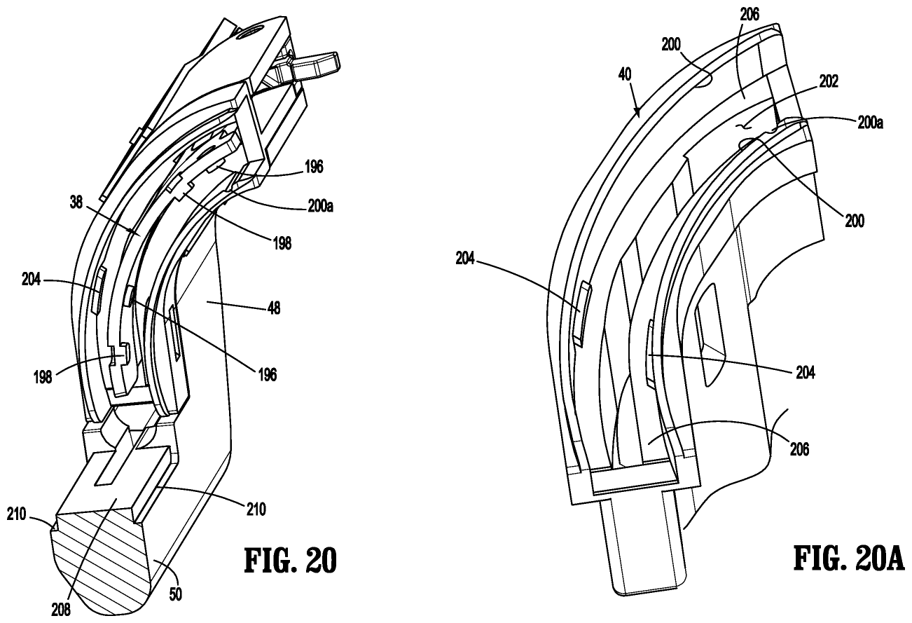

FIGS. 18 and 19 illustrate the interaction between the thrust bar 38 and the knife assembly 72 as the reload assembly 44 is coupled to the clamp slide assembly 40 (FIG. 3). As described above, when the reload assembly 44 is rotated onto the clamp slide assembly 44 (FIG. 3) in the direction indicated by arrow “A” in FIG. 18, the spaced protrusions 196 are received within the curved slot 188 and engage the curved wall 188a. In this position, the force distribution members 198 engage the curved abutment surface 190 of the knife holder 180 of the knife assembly 72.

FIGS. 20 and 20A illustrate the proximal portion of the end effector frame 42 and the distal portions of the clamp slide assembly 40 and the thrust bar 38 with the clamp slide assembly 40 and the thrust bar 38 in retracted positions received within the second transverse portion 48 of the end effector frame 42. As illustrated, the distal portion of the clamp slide assembly 40 includes side walls 200 that the cavity 54 (FIG. 3A) is in the form of a curved channel 202. Each of the side walls 200 includes a rib 204 that is received within the transversely extending slot 110 (FIG. 7) formed in the housing 74 of the cartridge assembly 62 of the reload assembly 44 (FIG. 6) when the reload assembly 44 is loaded onto the clamp slide assembly 40. The clamp slide assembly includes shelves 206 positioned on each side of the curved channel 202 for supporting the housing 74 of the cartridge assembly 62. The curved channel 202 of the clamp slide assembly 40 extends through the clamp slide assembly 40 and receives the thrust bar 38 such that the thrust bar 38 is movable in relation to the clamp slide assembly 40 within the curved channel 202. One of the side walls 200 defines a concavity 200a.

The longitudinal portion 50 of the end effector frame 42 includes a central portion 208 and longitudinal channels 210 positioned on each side of the central portion 208. The central portion 208 of the longitudinal portion 50 of the end effector frame 42 extends into the longitudinal recess 136 (FIG. 9) defined in the outer surface 132 of the longitudinal portion 114 of the reload frame 64 when the reload assembly 44 is loaded onto the clamp slide assembly 40 (FIG. 9).

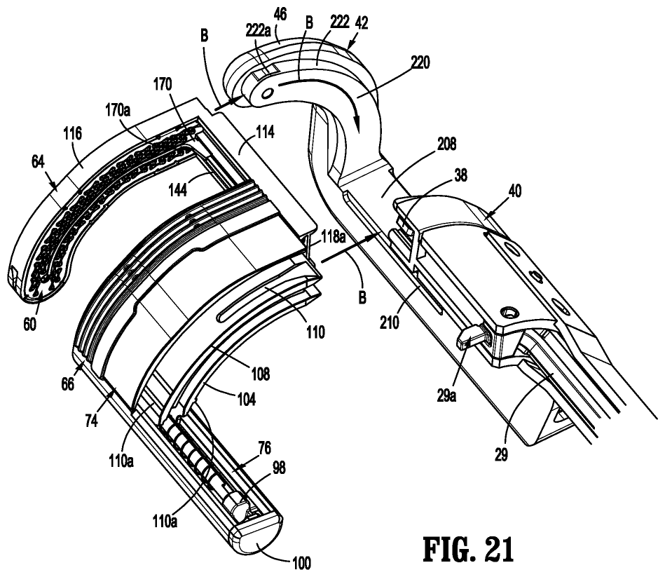

FIG. 21 illustrates the reload assembly 44 and the distal portion of the stapling device 10 (FIG. 1) including the end effector frame 42, the clamp slide assembly 40, and the thrust bar 38 as the reload assembly 44 is loaded onto the clamp slide assembly 40 and the end effector frame 42 in the direction indicated by arrows “B”. As illustrated, the first transverse portion 46 of the end effector frame 42 includes a raised platform 220 that is received in the curved recess 118a (FIG. 3) defined by the side walls 122 on the outer surface 118 of the transverse portion 116 of the reload frame 64 of the reload assembly 44. The raised platform 220 includes a side wall 222 that defines recesses 222a (only one is shown) that receives protrusions 226 (FIG. 6) formed on the side wall 120 of the transverse portion 116 of the reload frame 64. Simultaneously, the side walls 108 of the base member 104 of the housing 74 are slid into the curved channel 202 (FIG. 20A) of the clamp slide assembly 40 such that the ribs 204 (FIG. 20A) of the clamp slide assembly 40 are received in the transversely extending slot 110 formed in the base member 104 of the housing 74. When the housing 74 is slid fully into the channel 202 of the clamp slide assembly 40, the protrusion 110a is received in the concavity 200a (FIG. 20A) of the clamp slide assembly 40 to releasably secure the base member 104 of the housing 74 within the channel 202 of the clamp slide assembly 40.

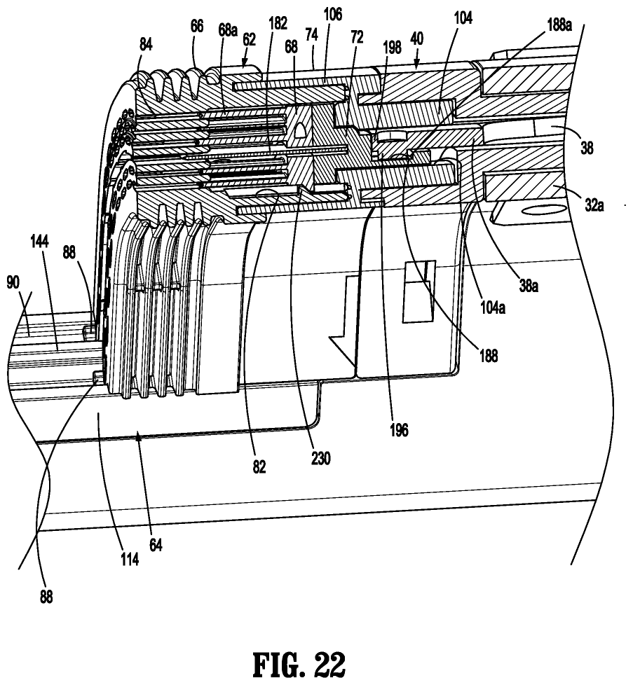

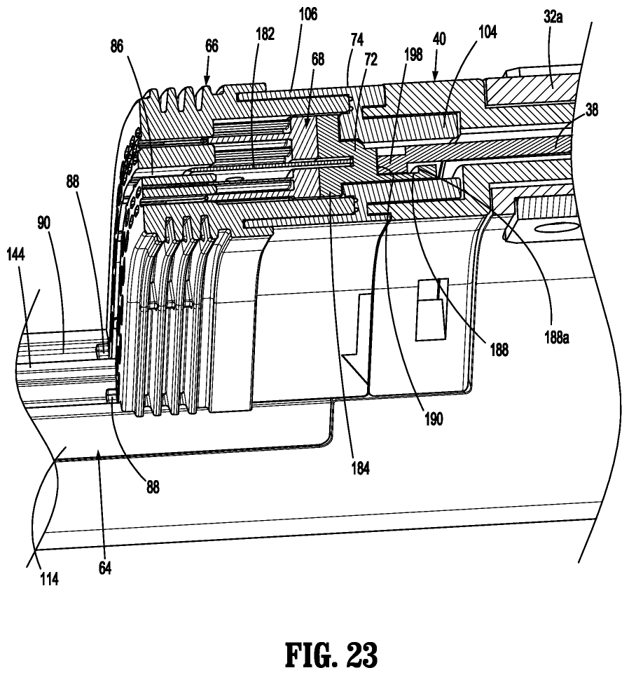

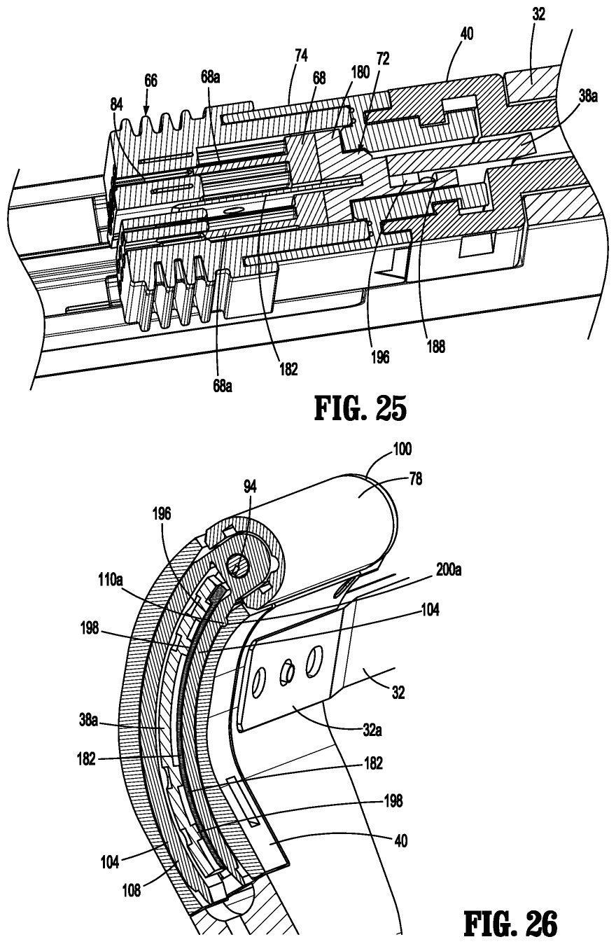

FIGS. 22-26 illustrate the reload assembly 44 secured to the end effector frame 42 with the stapling device in the open or unclamped position. As described above, as the reload assembly 44 is loaded onto the clamp slide assembly 40, the spaced protrusions 196 on the distal portion of the thrust bar 38 are received in the curved slot 188 of the knife holder 180 of the knife assembly 72 and the force distribution members 198 are in abutting relation to the curved abutment surface 190 of the knife holder 180. As illustrated, the cartridge body 66 of the cartridge assembly 62 includes a tab 230 that has an angled proximal surface that engages the pusher 68 to retain the pusher 68 in a retracted position within the cavity 82 of the cartridge body 66. Since the pusher 68 is engaged with a distal surface of the knife holder 180, the tab 230 also retains the knife assembly 72 in a retracted position. In the retracted position, the cutting blade 182 is recessed within the knife slot 86 of the cartridge body 66.

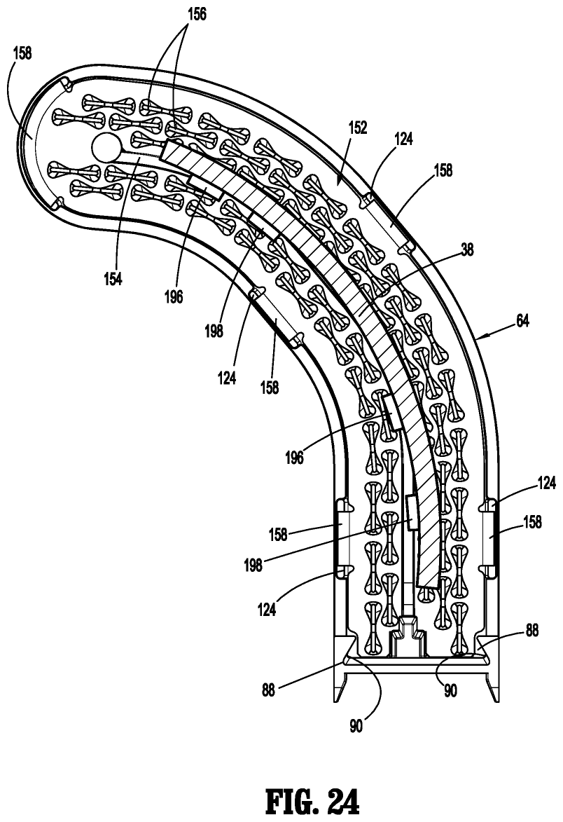

As described above, the distal portion 38a of the thrust bar 38 has a uniformly curved configuration, whereas the cartridge body 66 is defined by three linear portions that are angled in relation to each other. As such, the distal portion 38a of the thrust bar 38 is not uniformly positioned within the cartridge body 66 (FIG. 24). The addition of the force distribution members 198 on the distal portion 38a of the thrust bar balances the forces applied to the pusher 68 during firing of the stapling device 10 to improve staple formation.

The cartridge body 66 and the housing 74 of the cartridge assembly 62 are fixedly secured together and movable along the longitudinal portion 114 of the reload frame 64 in relation to the transverse portion 116 of the reload frame 64 and in relation to the anvil assembly 60 between retracted and advanced positions. As described above, the dove tails 88 on the cartridge body 66 are received in the dove tail channels 90 of the longitudinal portion 14 to guide movement of the cartridge assembly 62 between the retracted and advanced positions.

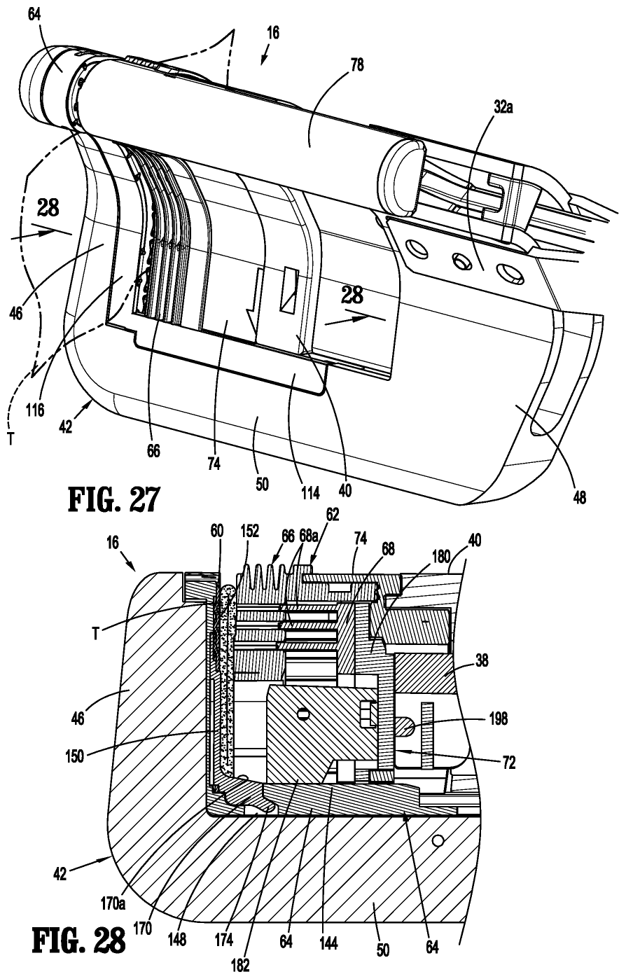

FIGS. 27 and 28 illustrate the end effector 16 in the clamped position. In the clamped position, the clamp slide assembly 40 is advanced within the gap 52 (FIG. 2) defined between the first and second transverse portions 46, 48 of the end effector frame 42 from a retracted position to an advanced position to move the cartridge assembly 62 into juxtaposed alignment with the anvil assembly 60 and clamp tissue “T” therebetween. In this position, the cutting blade 182 of the knife assembly 72 is in a retracted position recessed within the knife slot 86 (FIG. 6) of the cartridge body 66 and engaged with the raised rib 144 of the reload frame 64. As shown, the extension 170 of the cut plate 150 of the anvil assembly 60 is received within the cutout 148 of the reload frame 64 and the side edge of the cutting blade 182 is received on the raised rib 144 of the reload frame 64.

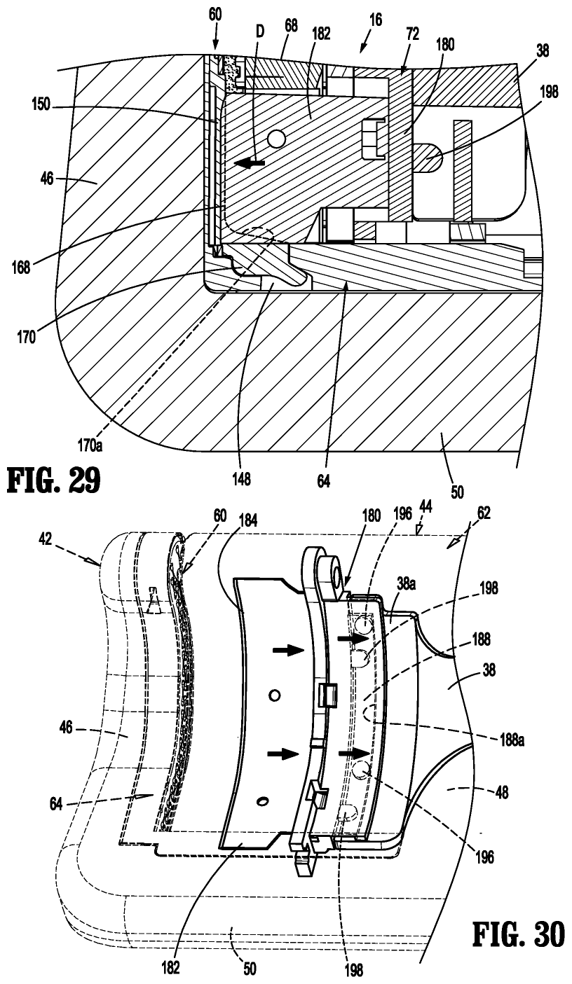

FIG. 29 illustrates the end effector 16 after the stapling device 10 (FIG. 1) is fired with the knife assembly 72 and the pusher 68 in advanced positions. When the stapling device 10 is fired, the thrust bar 38 moves distally in the direction of arrow “D” independently of the clamp slide 40 to advance the pusher 68 (FIG. 28) and the knife assembly 72 to eject the staples 70 (FIG. 6) from the cartridge body 66 and cut tissue (FIG. 28) clamped between the anvil 60 and the cartridge body 66. As the knife assembly 72 is advanced, the cutting blade 182 moves through the knife slot 86 of the cartridge body 66 and into the rib 168 of the cut plate 150. As the cutting blade 182 is advanced, the lower edge 182a of the cutting blade 182 cuts through the extension 170 of the cut plate 150. As described above, the inner surface 170a of the extension 170 of the cut plate 150 is angled upwardly towards the anvil 152. As such, as the cutting blade 152 is advanced towards the anvil 152, interference between the cutting blade 182 and the cut plate 150 increases and the cutting blade 152 cuts through a greater thickness of the extension 170. This increases the likelihood that tissue positioned adjacent the longitudinal portion 114 of the reload frame 64 at the edges of the cartridge assembly 62 is cleanly cut through by the cutting blade 182.

FIG. 30 illustrates the distal portion 38a of the thrust bar 38 and the knife assembly 72 as the thrust bar 38 is moved from its advanced position towards its retracted position. When the thrust bar 38 is retracted, the spaced protrusions 196 on the thrust bar 38 engage the wall 188a defining the proximal end of the curved slot 188 in the knife holder 180 to retract the knife holder 180 and the cutting blade 182 with the thrust bar 38. In the retracted position, the cutting blade 182 is returned to a positioned recessed within the cartridge body 66.

In aspects of this disclosure, the cutting plate 150 may be manufactured from a polypropylene material although other materials of construction are envisioned. It is also envisioned that the anvil 152 may be manufactured from stainless steel, e.g., stainless steel sheet metal although other materials of construction are envisioned.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A reload assembly comprising:
a reload frame including a longitudinal portion and a transverse portion, the longitudinal portion having a proximal end portion and a distal end portion, the transverse portion extending from the distal end portion of the longitudinal portion and having an outer surface and an inner surface;
an anvil assembly secured to the inner surface of the transverse portion, the anvil assembly including an anvil that defines a knife slot and staple forming depressions positioned on opposite sides of the knife slot; and
a cartridge assembly including a cartridge body, a pusher, a knife assembly, and staples, the cartridge body defining a cavity, a knife slot and staple receiving pockets positioned on each side of the knife slot, the pusher and the knife assembly received within the cavity of the cartridge body and movable between retracted and advanced positions, the pusher including fingers that are received within the staple receiving slots, one of the staples received within each of the staple receiving slots such that movement of the pusher from its retracted position to its advanced position ejects the staples from the staple receiving slots, the cartridge body and the longitudinal portion of the reload frame including interlocking structure that couples the cartridge body to the longitudinal portion of the reload frame while facilitating longitudinal movement of the cartridge assembly in relation to the anvil assembly.

2. The reload assembly of claim 1, wherein the interlocking structure includes dove tail shaped protrusions on the cartridge body and correspondingly shaped channels defined in the longitudinal portion of the reload frame.

3. The reload assembly of claim 1, wherein the interlocking structure includes rectangular protrusions on the cartridge body and correspondingly shaped channels defined in the longitudinal portion of the reload frame.

4. The reload assembly of claim 1, wherein the longitudinal portion of the reload frame includes an inner surface and an outer surface, the inner surface having a raised rib that extends inwardly from the inner surface of the longitudinal portion and is received within a channel defined by the cartridge body.

5. The reload assembly of claim 4, wherein the raised rib and the channel defined by cartridge body have stepped configurations.

6. The reload assembly of claim 1, wherein the anvil assembly includes a cut plate that is sandwiched between the transverse portion of the reload frame and the anvil.

7. The reload assembly of claim 6, wherein the anvil includes resilient brackets and the transverse portion of the reload frame includes slots, the resilient brackets received within the slots to secure the anvil and the cut plate onto the inner surface of the transverse portion of the reload frame.

8. The reload assembly of claim 7, wherein the transverse portion of the reload frame includes a tab that is positioned within each of the slots, and each of the resilient brackets defining an opening that receives one of the tabs to secure the anvil and the cut plate to the reload frame.

9. The reload assembly of claim 6, wherein the cut plate includes a rib that is aligned with the knife slot of the anvil.

10. The reload assembly of claim 9, wherein the rib extends through the knife slot of the anvil.

11. The reload assembly of claim 9, wherein the cut plate includes a proximal extension that extends proximally from the cut plate to a position proximally of the anvil and the knife assembly includes a cutting blade, the extension positioned on one end of the anvil and including an inner surface that is angled inwardly to a position inwardly of an end of the knife slot, the extension positioned to engage the cutting blade of the knife assembly when the knife assembly is moved to its advanced position.

12. The reload assembly of claim 6, wherein the cut plate is formed from polypropylene and the anvil is formed from stainless steel.

13. The reload assembly of claim 1, wherein the outer surfaces of the longitudinal and transverse portions of the reload frame define recesses that are configured to receive a portion of a surgical device.

14. A surgical stapling device comprising:
an elongate frame having a proximal portion and a distal portion and defining a channel;
a thrust bar positioned within the channel and movable between a retracted position and an advanced position, the thrust bar having a distal portion;
a clamp slide assembly positioned within the channel and having a proximal portion and a distal portion, the distal portion defining a clamp slide channel, the clamp slide assembly movable within the channel defined by the elongate frame between retracted and advanced positions, the distal portion of the thrust bar is positioned within the clamp slide channel;
an end effector frame secured to the distal portion of the elongate frame and including a first transverse portion, a second transverse portion, and a longitudinal portion, the longitudinal portion interconnecting the first transverse portion and the second transverse portion at spaced positions to define a gap between the first and second transverse portions, wherein the distal portion of the clamp slide assembly is positioned within the gap; and
a reload assembly including:
a reload frame including a longitudinal portion and a transverse portion, the longitudinal portion of the reload frame having a proximal end portion and a distal end portion, the transverse portion of the reload frame extending from the distal end portion of the longitudinal portion of the reload frame and having an outer surface and an inner surface;
an anvil assembly secured to the inner surface of the transverse portion of the reload frame, the anvil assembly including an anvil that defines a knife slot and staple forming depressions positioned on opposite sides of the knife slot; and
a cartridge assembly including a cartridge body, a pusher, a knife assembly, and staples, the cartridge body defining a cavity, a knife slot and staple receiving pockets positioned on each side of the knife slot, the pusher and the knife assembly received within the cavity of the cartridge body and movable between retracted and advanced positions, the pusher including fingers that are received within the staple receiving slots, one of the staples received within each of the staple receiving slots such that movement of the pusher from its retracted position to its advanced position ejects the staples from the staple receiving slots, the cartridge body and the longitudinal portion of the reload frame including interlocking structure that couples the cartridge body to the longitudinal portion of the reload frame while facilitating longitudinal movement of the cartridge assembly in relation to the anvil assembly between a retracted position and an advanced position, wherein the reload assembly is releasably supported on the end effector frame.

15. The surgical stapling device of claim 14, wherein the transverse portion of the reload frame includes an outer surface that defines a recess that receives the first transverse portion of the end effector frame when the reload assembly is loaded onto the end effector frame.

16. The surgical stapling device of claim 15, wherein the first transverse portion of the end effector frame includes a central portion and defines longitudinal channels on each side of the central portion, the central portion of the first transverse portion of the end effector frame received within the recess of the transverse portion of the reload frame.

17. The surgical stapling device of claim 14, wherein the knife assembly includes a knife holder and a cutting blade, the knife holder defining a slot.

18. The surgical stapling device of claim 17, wherein the distal portion of the thrust bar includes spaced protrusions, the spaced protrusions received within the slot of the knife holder when the reload assembly is loaded onto the end effector frame such that movement of the thrust bar from its advanced position towards its retracted position moves the knife assembly from its advanced position towards its retracted position.

19. The surgical stapling device of claim 18, wherein the distal portion of the thrust bar includes spaced force distribution members and the knife holder includes an abutment surface, the spaced force distribution members positioned along the distal portion of the thrust bar in engagement with the abutment surface of the thrust bar to balance firing forces applied by the thrust bar on the knife holder.

20. The surgical stapling device of claim 14, wherein the cartridge body is secured to a housing that is at least partly received within the clamp slide channel when the reload assembly is loaded onto the end effector frame, the distal portion of the clamp slide assembly including ribs positioned within the clamp slide channel, and the housing defining transversely extending slots, the transversely extending slots receiving the ribs when the reload assembly is loaded onto the end effector frame.
